(19) United States
(12) Patent Application Publication
(10) Pub. No.: US 2024/0342428 A1
(43) Pub. Date: Oct. 17, 2024

(54) INTERPRETATION BIAS MODIFICATION THERAPY USING A MOBILE DEVICE

(71) Applicant: The United States Government as represented by the Department of Veterans Affairs, Washington, DC (US)

(72) Inventors: Kirsten Dillon, Durham, NC (US); Jeffrey Hertzberg, Durham, NC (US)

(21) Appl. No.: 18/701,076

(22) PCT Filed: Oct. 13, 2022
(86) PCT No.: PCT/US22/46576

§ 371 Date: Apr. 12, 2024

Related U.S. Application Data
(60) Provisional application No. 63/255,381, filed on Oct. 13, 2021.

(51) Int. Cl. A61M 21/00

(52) U.S. Cl. CPC A61M 21/00; A61M 2021/0044

(57) ABSTRACT

Technologies are provided for implementing an interpretation bias modification (IBM) therapy using a mobile device. Some embodiments include a computing device that can initiate a session for interpretation bias modification (IBM) therapy, and can present, as part of the session, a statement describing an ambiguous anger-provoking situation. The computing device also can present, as part of the session, a second statement that comprises a non-threatening interpretation of such situation. That interpretation can be presented in natural language and missing at least one character. The computing device also can receive input defining one or more characters, and can determine that the one or more characters correspond to the at least one character missing in that interpretation. The computing device can then present, as part of the session, a comprehension question corresponding to the non-threatening interpretation, and can prompt selection of an answer to the comprehension question to reinforce the non-threatening interpretation.

### CROSS-REFERENCE TO RELATED APPLICATION

This application claims the benefit of and priority to U.S. Provisional Patent Application No. 63/255,381, filed Oct. 13, 2021, the contents of which application are hereby incorporated by reference herein in their entireties.

### BACKGROUND

According to cognitive models of anger, individuals with problematic anger have a tendency to interpret ambiguous interpersonal situations as hostile. For example, if someone bumps into them in a crowd they may be more likely to interpret this as an aggressive action than a mistake. In fact, hostile interpretation of situations has been identified as the first step in the elicitation of anger and subsequent aggression in multiple models of anger and aggression. A strong link between hostile interpretation biases and increased anger has been asserted in the literature, and researchers have demonstrated that hostile interpretation bias is linked to trait anger in children and adults.

Interpretation bias modification (IBM) techniques have been used to modify maladaptive interpretation biases that are theorized to cause and maintain anxiety and depression. IBM can be delivered via computer and can help participants to adopt more adaptive interpretational styles through repeated practice resolving ambiguous situations in a benign way.

### SUMMARY

It is to be understood that both the following general description and the following detailed description are illustrative and explanatory only and are not restrictive.

Embodiments of this disclosure include computing devices, methods, and computer-program products that, individually or in combination, can provide a mobile IBM therapy. More specifically, yet not exclusively, embodiments of this disclosure include a mobile device that has a memory device storing a mobile application in processor-executable form. Simply for the sake of nomenclature, the mobile application can be referred to as “Mobile Anger Reduction Intervention (MARI).” Execution of the mobile application by the mobile device can provide IBM therapy and many other related functionalities. The IBM therapy includes multiple treatment sessions administered over a defined period of time by means of the mobile device. Each treatment sessions includes an interactive training sequence of interactive user interfaces, where the training sequence includes a statement of an ambiguous anger-provoking situation, a non-threatening interpretation of that situation, and a question to reinforce such an interpretation. The interactive training sequence can be referred to as scenario. In order to traverse a scenario successfully, the mobile application causes the mobile device to present a challenge to complete a non-threatening interpretation correctly, and also to present, subsequently, a reinforcement question. A correct answer to the reinforcement question causes the mobile device to continue the IBM therapy by presenting an additional training session.

Additional elements or advantages of this disclosure will be set forth in part in the description which follows, and in part will be apparent from the description, or may be learned by practice of the subject disclosure. The advantages of the subject disclosure can be attained by means of the elements and combinations particularly pointed out in the appended claims.

This summary is not intended to identify critical or essential features of the disclosure, but merely to summarize certain features and variations thereof. Other details and features will be described in the sections that follow. Further, both the foregoing general description and the following detailed description are illustrative and explanatory only and are not restrictive of the embodiments of this disclosure.

### BRIEF DESCRIPTION OF THE DRAWINGS

The annexed drawings are an integral part of the disclosure and are incorporated into the subject specification. The drawings illustrate example embodiments of the disclosure and, in conjunction with the description and claims, serve to explain at least in part various principles, elements, or aspects of the disclosure. Embodiments of the disclosure are described more fully below with reference to the annexed drawings. However, various elements of the disclosure can be implemented in many different forms and should not be construed as limited to the implementations set forth herein. Like numbers refer to like elements throughout.

FIG. 1 illustrates an example of implementation of IBM therapy using a mobile device, in accordance with one or more embodiments of the disclosure.

FIG. 2A illustrates an example of a training scenario of IBM therapy implemented using a mobile device, in accordance with one or more embodiments of the disclosure.

FIG. 2B illustrates an example of another training scenario of IBM therapy implemented using a mobile device, in accordance with one or more embodiments of the disclosure.

FIG. 3 illustrates an example of yet another training scenario of IBM therapy implemented using a mobile device, in accordance with one or more embodiments of the disclosure.

FIG. 4A illustrates an example of a user interface (UI) of a mobile application that implements IBM therapy using a mobile device, in accordance with one or more embodiments of the disclosure.

FIG. 4B illustrates another example of the user interface (UI) of the mobile application that implements IBM therapy using a mobile device, in accordance with one or more embodiments of the disclosure.

FIG. 5 illustrates an example of another UI of the mobile application that implements IBM therapy using a mobile device, in accordance with one or more embodiments of the disclosure.

FIG. 6 illustrates an example of a user device that can implement an IBM therapy in accordance with one or more embodiments of the disclosure.

FIG. 7 illustrates an example of a method for implementing IBM therapy using a mobile device, in accordance with one or more embodiments of the disclosure.

### DETAILED DESCRIPTION

The disclosure recognizes and addresses the lack of IBM therapy using mobile devices. In particular, yet not exclusively, embodiments of this disclosure address the lack of IBM therapy to treat anger using a mobile device. Difficulty controlling anger is the most commonly reported reintegration concern among combat Veterans, especially those with a diagnosis of posttraumatic stress disorder (PTSD). In Veterans and other individuals, problematic anger is associated with numerous negative psychosocial outcomes, including poor functional outcomes (both social and occupational), family discord, aggression, road rage, and suicide risk. Anger can also impede successful outcomes from PTSD treatment. Thus, improved technologies for implementation of IBM therapy to treat anger may be desired. Existing treatments tend to be limited by low rates of engagement and high rates of dropout.

Embodiments of the disclosure address the implementation of IBM therapy to treat anger using a mobile device. Such an implementation of mobile health (mHealth) technology provides a low-cost approach to increase the reach of anger management treatments to high-need populations, such as Veterans afflicted by PTSD. Embodiments of this disclosure can provide a practical and effective mobile intervention for anger that can overcome at least some of the barriers that have kept Veterans and other individuals from engaging in, or benefitting from, anger management therapy. Further, embodiments of the disclosure can improve functional outcomes and community reintegration for Veterans and other individuals afflicted by PTSD.

One of the mechanisms associated with problematic anger and aggression is hostile interpretation bias; that is, a tendency to interpret ambiguous interpersonal situations as hostile. Embodiments of this disclosure can reduce hostile interpretation bias by providing an interactive environment via a mobile device. The mobile IBM therapy implemented using a mobile device in accordance with this disclosure can significantly reduce problematic anger and aggression, and also may improve functional outcomes.

As mentioned, embodiments of this disclosure include computing devices, methods, and computer-program products that, individually or in combination, can provide a mobile IBM therapy. Although the mobile IBM therapy of this disclosure is described with reference to anger therapy, the disclosure is not limited in that respect. Indeed, the principles and practical applications of this disclosure can be directed to other types of mobile IBM therapies, such as conflict resolution, executive function development (such as procrastination mitigation), or similar.

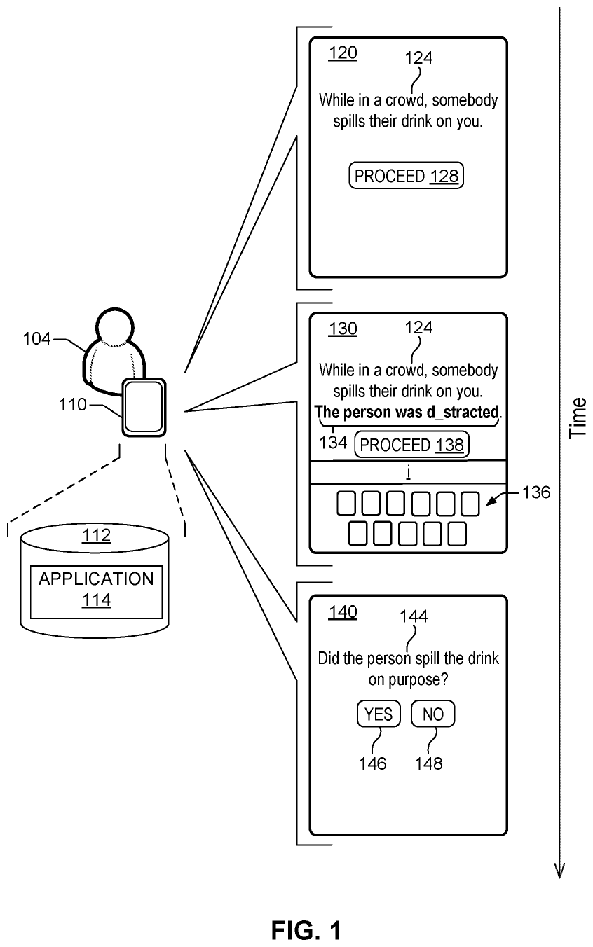

With reference to the drawings, FIG. 1 illustrates an example of implementation of IBM therapy using a mobile device 110, in accordance with one or more embodiments of the disclosure. Although the mobile device 110 is depicted as a smartphone, the disclosure is not limited in that respect. Indeed, the mobile device 110 can be any type of user device, such as an electronic-reader (e-reader) device, a tablet computer, a laptop computer, a portable gaming console, or similar device. Regardless of its type, the mobile device 110 includes computing resources (not shown) comprising, for example, one or more central processing units (CPU(s)), one or more graphics processing units (GPU(s)), memory devices, disk space, incoming bandwidth, and/or outgoing bandwidth, interface(s) (such as I/O interfaces or application programming interfaces (APIs), or a combination of both); controller devices(s); one or more power supplies; a display device and associated circuitry and components (lighting devices, control circuitry, conductive connectors, and the like), a combination of the foregoing; and/or similar resources.

The mobile device 110 can execute a mobile application 114 to implement an IBM therapy. The mobile application 114 can be retained in one or multiple memory devices 112 (which can be generically referred to as memory 112). The IBM therapy can be implemented over a defined time interval, executing a treatment session periodically within the defined time interval. In one example, the defined time period can be four weeks and the periodicity of the treatment sessions can be one day. In other words, the implementation of the IBM therapy can include daily treatment session over four weeks. Accordingly, in that example, the IBM treatment can include 28 treatment sessions. The IBM treatment is, of course, not limited to 28 treatment sessions over four weeks. The IBM treatment includes multiple treatment sessions, distributed over the defined time interval.

Regardless of periodicity, each treatment session contains a defined number Σ (a natural number) of training scenarios, and can span up to a second defined time interval. In some cases, each treatment session spans up to about 10 minutes and includes Σ=42 training scenarios. For purposes of illustration, a scenario can be embodied in a sequence of an ambiguous anger-provoking statement, a non-threatening interpretation of the statement, and a reinforcement question. Training scenarios capture a wide range of different themes that can be anger-provoking. Simply as an illustration, themes can include physical aggression, driving situations, irritating traits of others, thinking you are being ignored by others, feeling argued with or criticized, thinking someone is stealing from you, having people block you from social situations (in-person or online), thinking that others have hostile feelings, feeling disrespected, thinking that people will not help you, thinking that others do not appreciate you, thinking that situations are unfair, a combination of the foregoing, or similar themes. The training scenarios can be reviewed PTSD experts and/or individuals with PTSD to confirm that the content presented by the scenarios is relevant to a population of participants (such as Veterans afflicted by PTSD).

More specifically, as is illustrated in FIG. 1, the mobile device 110 can initiate a treatment session for an IBM therapy by executing, or continuing to execute, the mobile application 114. Within the treatment session, the mobile device 110 can present a user interface 120 that includes a first statement 124 describing an ambiguous anger-provoking situation. To that end, in response to execution of the mobile application 114, the mobile device 110 can cause a display device to present the user interface 120. The display device can be integrated into the mobile device 110. As is illustrated in FIG. 1, in one example scenario, the first statement 124 can be “While in a crowd, somebody spills their drink on you.” As part of the IBM therapy, a participant 104 that uses the mobile device 110 can be instructed to imagine themselves in the described situation. Such an instruction can be provided prior to presenting the user interface 120. To that end, the mobile device 110 can present a description of the IBM therapy and/or related instructions in a user interface (not depicted in FIG. 1) prior to presenting the user interface 120.

The user interface 120 also can include a selectable user interface (UI) element 128. Selection of the selectable UI element 128 can cause the mobile device 110 to present a user interface 130 as part of the treatment session, during execution of the mobile application 114. Such a selection can be accomplished by means of a user interaction with the mobile device 110. For purposes of illustration, in this disclosure, a user interaction can include a screen tap or swipe, a screen click, or similar. The display device integrated into the mobile device 110 can present the user interface 130. In some embodiments, rather than relying on the selectable visual element 128, an interaction with the mobile device 110, such as a screen tap, can cause the presentation of the user interface 130.

The user interface 130 includes a second statement that includes a non-threatening interpretation 134 of the ambiguous anger-provoking scenario conveyed by the first statement 124. The second statement is subsequent to, and in some cases also includes, the first statement 124. The mobile device 110 presents the non-threatening interpretation 134 in natural language and in incomplete form, with at least one letter missing. As is illustrated in FIG. 1, the non-threatening interpretation 134 can be “The person was d_stracted” and, as is shown, misses the letter “i.” Besides missing a letter, in some cases, a non-threatening interpretation can lack at least one number or at least one other type of character, or both.

The user interface 130 also can include multiple selectable visual elements 136 that permit the participant 104 to fill in the missing letter(s) in the non-threatening interpretation 134. In some embodiments, a layout of the multiple selectable visual elements 136 can form a graphical keyboard or a portion thereof. In other embodiments, the layout of the multiple selectable visual elements 136 can be different from a graphical keyboard. For instance, the layout can be an array of areas, each area in the array corresponding to a selectable visual element.

As such, regardless of the spatial structure of the layout, the multiple selectable visual elements 136 can permit the mobile device 110 to receive input data defining one or more characters. In cases where a graphical keyboard or a portion thereof is not presented, the array of areas provides multiple-choice scenario with respect to selection of the one or more characters. In some instances, the one or more characters do not correctly complete the non-threatening interpretation 134. Hence, the mobile device 110 can continue presenting the second statement having the first statement 124 and the non-threatening interpretation 134. In addition, or in some embodiments, the mobile device 110 can present a message including visual elements or aural elements, or both, that prompt the participant 104 to enter another character. For instance, the message can be “Please try again.” The message can be embodied in, or can include, a push notification that overlays the user interface 130, in some cases. Further, or in yet other embodiments, the mobile device 110 can redraw the multiple selectable element 136 with a lesser number of elements as erroneous attempts to complete the non-threatening interpretation 134 accumulate. In that way, the mobile application 114 causes the mobile device 110 to redraw the user interface 130 with a lesser complexity as erroneous attempts accumulate, thus converging towards a correct completion of the non-threatening interpretation 134.

In other instances, the character(s) defined by the input data can correspond to the at least one letter missing in the non-threatening interpretation 134. In those instances, the non-threatening interpretation 134 can be correctly completed, e.g., the word “distracted” is formed, and a benign interpretation is assigned to the ambiguous anger-provoking situation conveyed by the first statement 124. In some cases, still within the session, the mobile device 110 can present a congratulatory message in response to the correct word being formed. The message can be presented in an overlay section on the user interface 130. In one example, the overlay section can include text and/or graphics conveying a congratulation, such as “Good job!” or “Well done!” In further response, or an as alternative, within the session, the mobile device 110 can determine that the character(s) defined by the input data received by the mobile device 110 correspond to the at least one letter missing in the non-threatening interpretation 134. The mobile device 110 can permit reinforcement of the non-threatening interpretation 134 in response to such a determination, as part of the treatment session, during execution of the mobile application 114. Such an interpretation can be reinforced by requiring the participant 104 to correctly answer “Yes” or “No” to a comprehension question corresponding to the non-threatening interpretation 134.

Accordingly, in some configurations, the mobile device 110 can present a selectable visual element 138 in response to receiving the correct character(s) that complete the non-threatening interpretation 134. Selection of the selectable visual element 138 can cause the mobile device 110 to present a user interface 140 as part of the treatment session, during execution of the mobile application 114. Such a selection can be accomplished by means of a user interaction with the mobile device 110. The display device integrated into the mobile device 110 can present the user interface 140. The user interface 140 includes a comprehension question 144 corresponding to the non-threatening interpretation 134. The mobile device 110 also can prompt selection of an answer to the comprehension question 144 to reinforce the non-threatening interpretation. Thus, in some configurations, the user interface 140 also can include a first selectable visual element 146 and a second selectable visual element 148 corresponding to respective answers to the comprehension question 144. Only one of the first selectable visual element 146 or the second selectable visual element 148 corresponds to a correct answer. In the example scenario illustrated in FIG. 1, “No” is the correct answer.

The mobile device 110 can receive input data representing an answer to the comprehension question 144. In some cases, the mobile device 110 can then determine that the answer is correct and, thus, reinforces the non-threatening interpretation 134. The correct answer can cause the mobile device 110 to present a congratulatory message. The message can be presented in an overlay section on the user interface 140. In one example, the overlay section can include text, graphics, speech, and/or sounds conveying a congratulation, such as “Good job!” or “Well done!” In addition, or in some embodiments, the correct answer causes the mobile application 114 to direct the mobile device 110 to determine if the treatment session has been completed. In response to a determination that the answer is incorrect, the mobile application 114 can cause the mobile device 110 to continue the treatment session by presenting another training scenario. In some cases, an incorrect answer can cause the mobile application 114 to direct the mobile device 110 to present a message including visual elements or aural elements, or both, that prompt the participant 104 to provide another answer. For instance, the message can be “Please try again.” The message can be embodied in, or can include, a push notification that overlays the user interface 140, in some cases. Further, or in yet other cases, in response to the negative determination, the mobile application 114 also can cause the mobile device 110 to determine if the treatment session has been completed. In other words, there are embodiments in which regardless of whether or not a correct answer is received by the mobile application 114, the mobile application 114 directs the mobile device 110 to determine if the treatment session has been completed (e.g., Σ training scenarios have been presented).

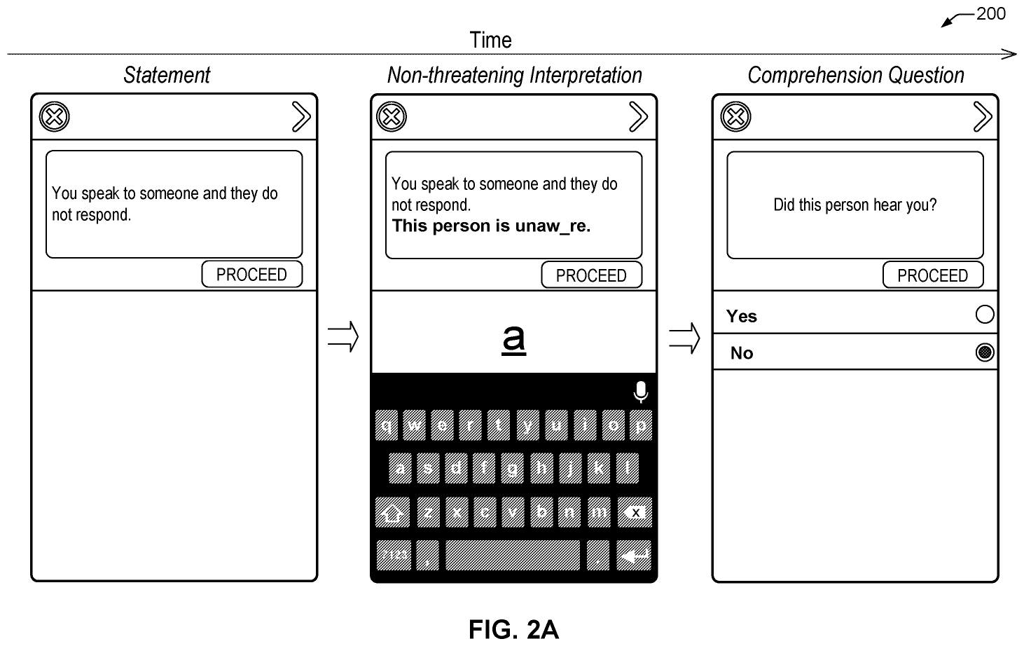

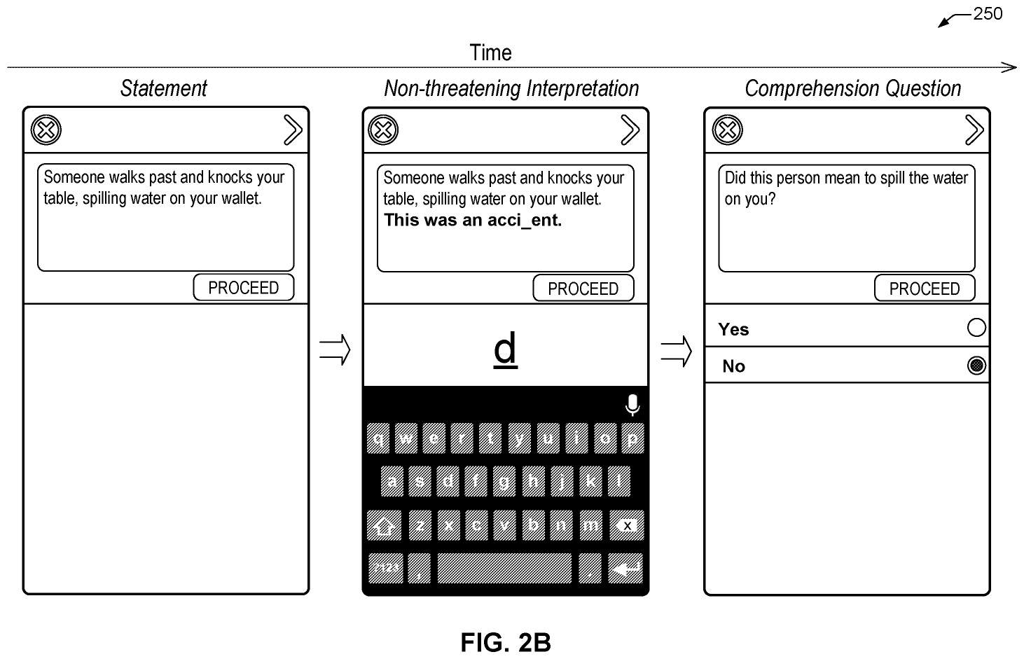

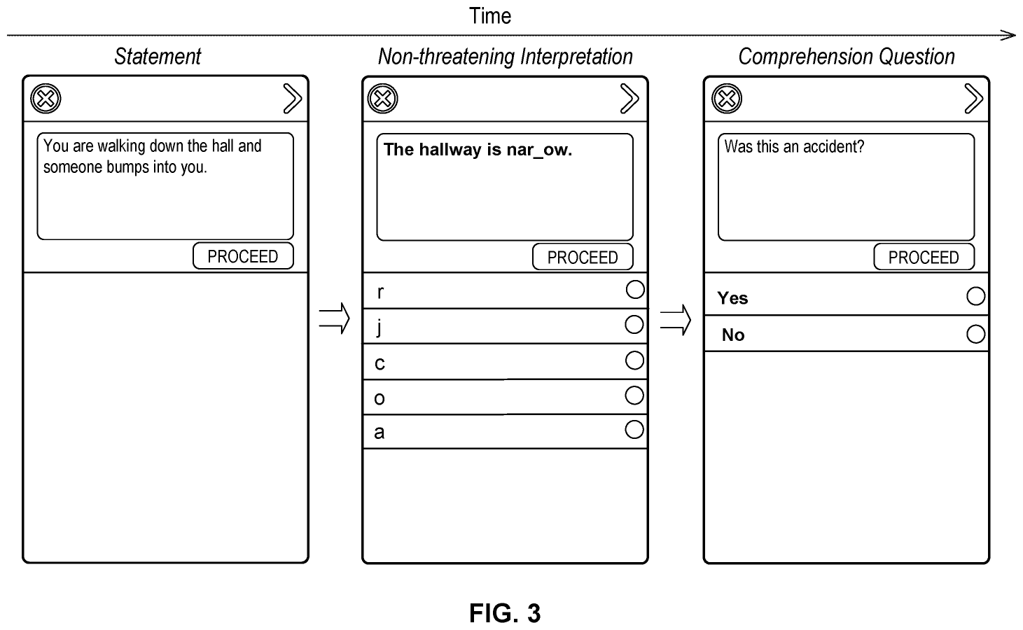

As an example, the mobile device 110 can present the training scenario 200 shown in FIG. 2A or the training scenario 250 shown in FIG. 2B. In those training scenarios, the selectable visual elements 136 form a QWERTY graphical keyboard. As another example, the mobile device 110 can present the training scenario shown in FIG. 3, where the selectable visual elements 136 for an array of five rectangular areas, each area corresponding to a selectable character option. Selection can be effected by checking a circular indicium (e.g., a radio-button elements).

The mobile application 114 is configured (e.g., programmed, or programmed and built) to avoid repeating scenarios across training sessions. That is, the mobile application 120 can deliver distinct training scenarios in each treatment session. Thus, in one example, none of the training scenarios is repeated across the 28 treatment sessions that can constitute an IBM therapy. To avoid repetition of training scenarios, the mobile application 120 can be configured to use N unique training scenarios, where Nis a natural number much greater than the M·Σ. Here, M is the number of treatment sessions pertaining to an IBM therapy. In one embodiment, N=1,176.

In those training scenarios, statements that describe ambiguous anger-provoking situations have reading levels that are accessible to most, if not all, participants in IBM therapy in accordance with this disclosure. In some example configurations, the reading level can be 6^{th}-grade reading level or less. Thus, the sequence of Us presented during a treatment session include content at a defined reading level that can be satisfactory to a participant (e.g., a Veteran afflicted by PTSD).

In response to a determination that the treatment session has ended—e.g., Σ training scenarios have been presented—the mobile application 114 can cause the mobile device 110 to implement one or several post-session operations. In some embodiments, a post-session operation can include providing points for completion of a treatment session and/or badges of achievement. As such, the mobile device 110 can provide rewards for completing the treatment session. The rewards can be provided by executing, or continuing to execute, the mobile application 114. More specifically, the mobile device 110 can generate a token representing completion of the session. The mobile device 110 can then assign the token to a user profile corresponding to the mobile application 114. That user profile can be specific to a participant in IBM therapy, such as the participant 104. A token can be embodied in, or can include, for example, a data record defining one or more multiple points. In another example, a token can be embodied in a data record defining a badge of achievement. Such a data record can include imaging data defining a graphical asset (e.g., a still image or an animation) and/or formatting data defining the manner of displaying the badge in a user interface.

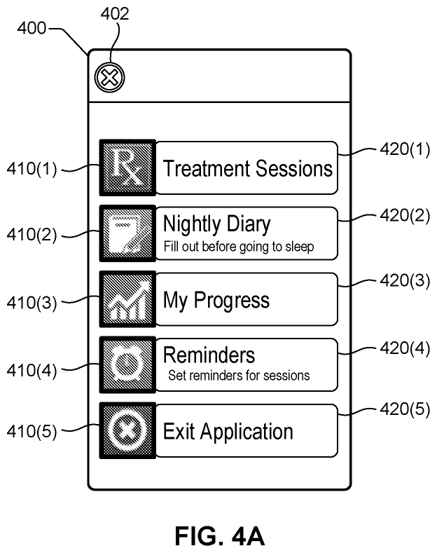

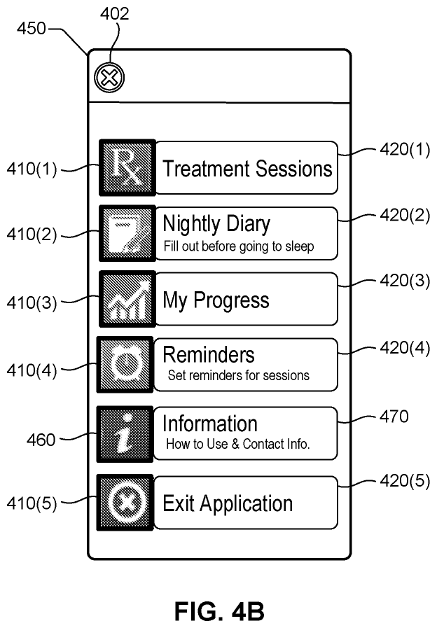

In addition to implementing IBM therapy using the mobile device 110, the mobile application 114 can be configured to provide several functionalities in response to execution by the mobile device 110. Some of those functionalities can be accessed in response to user-interaction with the mobile device 110. As mentioned, a user interaction can include a screen tap or swipe, a screen click, or similar, for example. That user interaction can permit specifying a selection of a functionality. As is illustrated in FIG. 4A, execution of the mobile application 114 can cause the mobile device 110 to present a user interface 400 displaying a menu of functionalities provided by mobile application 114. The user interface 400 also includes a selectable visual element 402 that, in response to being selected, causes the mobile device 110 to continue executing the mobile application 114 in a background thread.

The menu of functionalities can be embodied in multiple icons and respective selectable visual elements. The multiple icons can include selectable icons or non-selectable icons, or a combination thereof. Each selectable visual element corresponding to an icon can have markings identifying functionality accessible via the selectable visual element. One or more of the multiple icons can be presented according to a color palette having cool colors that may alleviate stress. The background of the user interface 400 also can be colored according to one or more colors from such a color palette. Selection of a first one of the selectable visual elements causes the mobile device 110 to provide a first one of the multiple functionalities, and selection of a second one of the selectable visual elements causes the mobile device 110 to provide a second one of the multiple functionalities. Again, such a selection can be accomplished by means of a user interaction with the mobile device 110.

As is illustrated in FIG. 4A, the multiple icons include a first icon 410(1), a second icon 410(2), a third icon 410(3), a fourth icon 410(4), and a fifth icon 410(5). The first icon 410(1) has a corresponding selectable visual element 420(1) labeled “Treatment Sessions.” Selection of the selectable visual element 420(1) causes the mobile device 110 to implement a treatment session in accordance with aspects described herein. Such a selection can be accomplished by means of a user interaction with the mobile device 110.

The second icon 410(2) has a corresponding selectable visual element 420(2) that is labeled “Nightly Diary” and prompts the participant 104 to complete a task before going to sleep. The disclosure is not limited in that respect. Indeed, in some embodiments, the second icon 410(2) can be labeled “Diary” and can prompt completion of the task at another period of the day. Selection of the selectable visual element 420(2) causes the mobile device 110 to implement a survey or creation of a diary entry where the participant 104 can report one or more of the following, for example: (1) what their stress level was that day on a defined scale, e.g., level 0 (no stress) to level 10 (highly stressful day); (2) how angry they felt that day on a defined scale, e.g., level 0 (no anger) to 10 (extreme anger); (3) how happy they felt that day on a defined scale, e.g., level 0 (unhappy) to level 10 (delighted); (4) how content they felt that day on a defined scale on a defined scale, e.g., level 0 (not content at all) to level 10 (highly content); (5) how much pain they experienced that day on a define scale, e.g., level 0 (no pain) to level 10 (excruciating pain); (6) how helpful they found the mobile application 114 that day on a defined scale, e.g., level 0 (useless) to level 10 (highly useful); (7) whether the treatment sessions made them think or feel differently about anything that happened to them that day; or (8) if yes to question (7), the participant 104 can briefly explain what happened that day. In some embodiments, the survey or diary entry can include fewer or more than eight survey items.

In some embodiments, to implement the survey, the mobile device 110 can present a sequence of user interfaces. Each user interface in the sequence corresponds to an item in the survey (or, in some cases, an item in the diary entry). Further, each user interface in the sequence can include a selectable pane having a defined element to receive data responsive to the item of the survey. In other embodiments, to implement the survey, the mobile device 110 can present a single user interface including a selectable pane conveying the survey as a whole, where the selectable pane includes multiple UI elements to receive data responsive to respective items of the survey. The user interface can include selectable navigation elements that can control the amount of content that is visible in the user interface. For instance, the navigation elements can permit scrolling up and down the content within the user interface. In addition, or in some embodiments, the amount of content that is visible in the user interface can be controlled with a gesture, such as a swipe or a sustained touch along an upward or downward direction.

In some embodiments, execution of the mobile application 114 can cause the mobile device 110 to prompt the participant 104 each night to complete a survey or create a diary entry. To that end, the mobile application 114 can cause the mobile device 110 to direct a display device to present a push notification (or another type of message) including content prompting the participant 104 to complete the survey or create the diary entry.

The third icon 410(3) shown in FIG. 4A has a corresponding selectable visual element 420(3) that is labeled “My Progress.” Selection of the selectable visual element 420(3) causes the mobile device 110 to provide a record of treatment sessions completed by a participant using the mobile application 114. That record can permit the participant (e.g., participant 104) to keep track of how many sessions the participant has completed. In some configurations, the participant also can see participant's performance across treatment sessions in the form of a graph (e.g., number of scenarios resolved, time spent, or similar). Such a selection can be accomplished by means of a user interaction with the mobile device 110. In order to track performance, the mobile application 114 can uses time stamps for defined events related to a treatment session so that treatment time and completion time (in terms of hours, minutes, and seconds, and/or date, for example) for the participant 104 can be determined. A defined event can be initiation of a treatment session, election to proceed from a statement describing an ambiguous anger-provoking situation, completion of a non-threatening interpretation of the situation, response to a reinforcement questions, or similar, for example.

When the mobile application 114 is first executed by the mobile device 110, the mobile device 110 can present a prompt to select times of day that can be satisfactory (most convenient, second most convenient, etc.) to complete treatment sessions. In addition, the mobile application 114 can provide functionality to configure reminders to complete treatment sessions. Specifically, the fourth icon 410(4) can permit configuring a session reminder to complete treatment sessions. The fourth icon 410(4) has a corresponding selectable visual element 420(4) that is labeled “Reminders” and includes text (or other markings, in some cases) prompting the participant 104 to configure such a session reminder. Selection of the selectable visual element 420(4) causes the mobile device 110 to present a user interface in response to selection of the selectable visual element 420(4). That user interface includes second selectable visual elements to configure the session reminder. Such a selection can be accomplished by means of a user interaction with the mobile device 110.

The fifth icon 410(3) shown in FIG. 4A has a corresponding selectable visual element 420(5) that is labeled “Exit Application.” Selection of the selectable visual element 420(5) causes the mobile device 110 to terminate execution of the mobile application 114.

Other layouts of menu of functionalities also are contemplated. In addition, one or more other functionalities also can be implemented. As an illustration, execution of the mobile application 114 can cause the mobile device 110 to present the UI 450 shown in FIG. 4B. The UI 450 provides an example of an alternative layout and additional functionality. The alternative layout is similar to the layout of menus of functionalities that is included in the UI 400 (FIG. 4A). The alternative layout includes an additional icon 460 relative to the icons 410(1) to 410(5) in the UI 400. The additional icon 460 can permit accessing a description of how to use the mobile application 114 and/or contact information of a support team, a telehealth therapist, or the like, related to the mobile application 114. The description can include, for example, an explanation of the treatment rationale and a reminder of the suggested treatment schedule (e.g., at least five times a week for four weeks). To permit access to the description and/or the contact information, the additional icon 460 has a corresponding selectable visual element 470 that is labeled “Information.” The selectable visual element 470 includes text (and/or other markings, in some cases) conveying the type of information that can be accessed via the selectable visual element 470.

In some embodiments, selection of the selectable visual element 470 causes the mobile device 110 to present a user interface in response to selection of the selectable visual element 470. The user interface that is presented can include text, graphics, hyperlinks, and/or other markings (selectable or otherwise) that convey a description of how to use the mobile application 114 and/or contact information of a support team, a telehealth therapist, or the like. In addition, or in other embodiments, selection of the selectable visual element 470 causes the mobile device 110 to present audible signal and/or aural elements in response to selection of the selectable visual element 470. The aural elements or the audio, or a combination of both, can convey the description of how to use the mobile application 114 and/or contact information of the support team, the telehealth therapist, or the like. The audible signal can be representative of speech delivered by a natural speaker or a bot speaker.

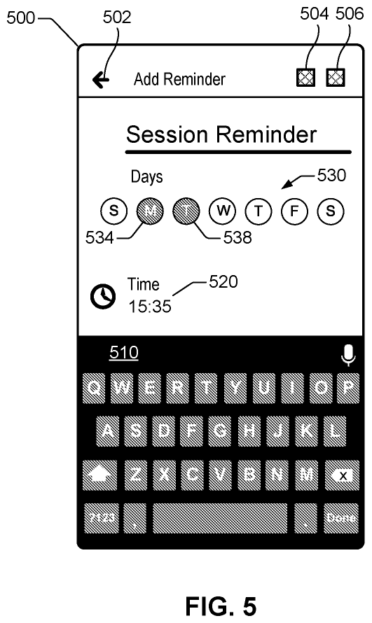

An example of such a user interface is illustrated in FIG. 5. The user interface 500 includes selectable indicia representing a keyboard 510. Although the keyboard 510 is shown as a QWERTY keyboard, the disclosure is not limited in that respect and other types of keyboards can be presented. Selection of the one or more of the selectable indicia can fill in a field 520 to configure a time of the reminder. Such a selection can be accomplished by means of a user interaction with the mobile device 110. The user interface 500 also can include second selectable indicia 530 representing the days of the week. As is shown in FIG. 5, the second selectable indicia 530 form a row, and each indicium includes a letter (“S,” “M,” “T,” “W,” “T,” “F,” or “S,”) representing a particular day of the week. The leftmost indicium represents Sunday (S) and the rightmost indicium represents Saturday (S), in an orientation dictated by Western reading/writing orientation. An indicium that has been selected to represent a day having a session reminder can be formatted differently from another indicium that has not been selected to represent a day having a session reminder. In the user interface 500, a first selectable indicium 534 and a second selectable indicium 538 have been selected to represent days having session reminders at the time shown in the field 520.

The user interface 500 also include a selectable visual element 502 that, in response to being selected, cause the mobile device 110 to return to the menu of functionalities of the mobile application 114 by again presenting the user interface 400 (FIG. 4A) or, in some cases, the user interface 450 (FIG. 4B). The user interface 500 also can present a selectable visual element 504 that, in response to being selected, can cause the mobile device 110 to clear an extant reminder. For instance, selection of the selectable visual element 504 can result in the field 520 being cleared, and indicium 534 and indicium 538 being deselected. Further, the user interface 500 also can include a selectable visual element 506 that, in response to being selected, causes the mobile device 110 create a data record in the memory 112, where the data record is indicative of a reminder that has been configured. Such a selection can be accomplished by means of a user interaction with the mobile device 110.

The mobile application 114 can be configured (e.g., programmed, or programmed and built) to provide several additional functionalities. One or more of those additional functionalities can enhance participants' engagement with the mobile application 114 and access to IBM therapy using a mobile device having the mobile application 114 stored therein. Specifically, the mobile application 114 can be configured to direct the mobile device 110 to cause presentation of messages periodically to the participant 104. The mobile device 110 can cause a display device integrated therein to present such messages. Examples of types of messages that can be presented include push notification, short message service (SMS) message, multimedia messaging service (MMS), email, and the like. In one configuration, execution of the mobile application 114 can direct the mobile device 110 to cause the display device to present a message periodically, where the message prompts the participant 104 to maintain a current frequency of treatment sessions for IBM therapy. As an illustration, when a participant adheres to a suggested treatment schedule (e.g., at least five treatment sessions per week), the participant can receive a congratulatory push notification at the end of that week. That push notification can include content that encourages the participant to keep up the frequency of sessions. Such content can include, for example, text, a still picture, an animation, or similar content).

In addition, or in another configuration, execution of the mobile application 114 can direct the mobile device 110 to cause the display device to present a message after a period of inactivity. To that end, execution of the mobile application 114 can cause the mobile device 110 to determine if a next treatment session for IBM therapy has not been initiated for a defined time interval (e.g., one day, two days, three days, four days, five days, a week). A positive determination causes presentation, by the mobile device 110, of a message prompting the participant 104 to initiate the next treatment session. As an illustration, participants who have not used the mobile application 114 for several days can be presented with a push notification including content that reminds a participant to do treatment sessions and/or second content providing suggestions for how to increase adherence (e.g., blocking out 10 minutes each day, setting up reminders, or similar).

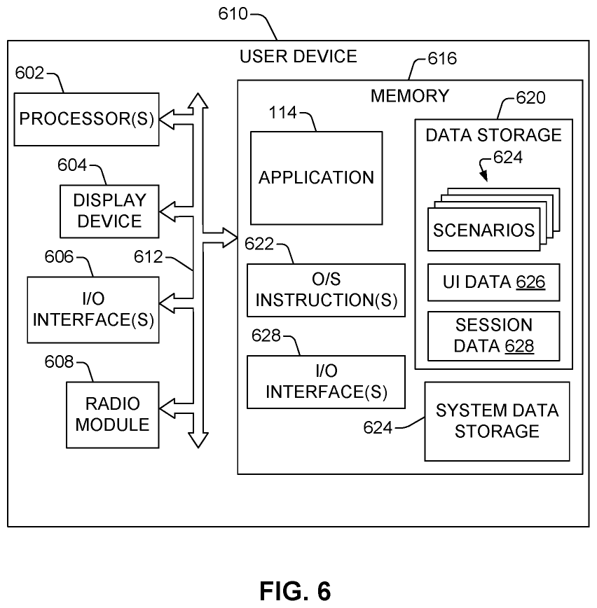

FIG. 6 is a block diagram of an example of a user device 610 that can operate in accordance with one or more aspects of the disclosure. As such the user device 610 can implement an IBM therapy according to one or more embodiments of this disclosure. The user device 610 can embody, or can constitute, the mobile device 110 (FIG. 1) in some cases. As is illustrated in FIG. 6, the user device 610 can include one or more memory devices 616 (referred to as memory 616). The memory 416 can have processor-accessible instructions encoded thereon. The processor-accessible instructions can include, for example, program instructions that are computer readable and computer-executable.

The user device 610 also can include one or multiple input/output (I/O) interfaces 606, a display device 604, and a radio module 608. A bus architecture 612 can functionally couple two or more of those functional elements of the user device 610. The bus architecture 612 represents one or more of several types of bus architectures, including a memory bus or memory controller, a peripheral bus, an accelerated graphics port, and a processor or local bus using any of a variety of bus architectures. As an example, such architectures can comprise an ISA bus, an MCA bus, an EISA bus, a VESA local bus, an AGP bus, and a PCI, a PCI-Express bus, a PCMCIA bus, a USB bus, or the like.

Functionality of the user device 110 can be configured by computer-executable instructions (e.g., program instructions or program modules) that can be executed by at least one of the one or more processors 602. A subset of the computer-executable instructions can be embody the mobile application 114. Such a subset can be arranged in a group of software components. A software component of the group of software components can include computer code, routines, objects, components, data structures (e.g., metadata objects, data object, control objects), a combination thereof, or the like, that can be configured (e.g., programmed) to perform a particular action or implement particular abstract data types in response to execution by the at least one processor.

Thus, the mobile application 114 can be built (e.g., linked and compiled) and retained in processor-executable form within the memory 616 or another type of machine-accessible non-transitory storage media. The mobile application 114 in processor-executable form, for example, can render the user device 610 (or any other computing device that contains the mobile application 114) a particular machine for mobile IBM therapy, among other functional purposes. The group of built software components that constitute the processor-executable version of the mobile application 114 can be accessed, individually or in a particular combination, and executed by at least one of the processor(s) 602. In response to execution, the mobile application 114 can provide the functionality described herein in connection with IBM therapy. Accordingly, execution of the group of built software components retained in the memory 616 can cause the user device 610 to operate in accordance with aspects described herein.

Data and processor-accessible instructions associated with specific functionality of the user device 610 can be retained in the memory 616. At least a portion of such data and at least a subset of those processor-accessible instructions can permit implementation of an IBM therapy in accordance with aspects described herein. In one aspect, the processor-accessible instructions can embody any number of components (such as program instructions and/or program modules) that provide specific functionality in response to execution by at least one of the processor(s) 602. In the subject specification and annexed drawings, memory elements are illustrated as discrete blocks; however, such memory elements and related processor-accessible instructions and data can reside at various times in different storage elements (registers, files, memory addresses, etc.; not shown) in the memory 616.

The memory 616 can include data storage 620 that can comprise a variety of data, metadata, or both, associated with an IBM therapy in accordance with aspects described herein. As is illustrated in FIG. 6, the data storage 620 can include data defining multiple training scenarios 624. The multiple training scenarios 624 can embody, or can include, the N training scenarios described above. The data storage 620 also can include UI data 626 defining various types of formatting attributes (layout, font, font size, color, etc.) for the user interfaces presented during a treatment session and other user interfaces corresponding to other functionalities of the mobile application 114. The data storage 620 can further include session data 628 including various data identifying defined events associated with treatment sessions pertaining to an IBM therapy, for example.

Memory 616 can be embodied in a variety of computer-readable media. Example of computer-readable media can be any available media that is accessible by a processor in a computing device (such as one processor of the processor(s) 602) and comprises, for example volatile media, non-volatile media, removable media, non-removable media, or a combination the foregoing media. As an example, computer-readable media can comprise “computer storage media,” or “computer-readable storage media,” and “communications media.” Such storage media can be non-transitory storage media. “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, DVD or other optical storage, magnetic cassettes, magnetic tape, magnetic disk storage or other magnetic storage devices, or any other medium which can be utilized to store the desired information and which can be accessed by a computer or a processor therein or functionally coupled thereto.

Memory 616 can comprise computer-readable non-transitory storage media in the form of volatile memory, such as RAM, EEPROM, and the like, or non-volatile memory such as ROM. In one aspect, memory 616 can be partitioned into a system memory (not shown) that can contain data and/or program modules that enable essential operation and control of the user device 110. Such program modules can be implemented (e.g., compiled and stored) in memory elements 622 (referred to as O/S instruction(s) 622) whereas such data can be system data that is retained in memory element 624 (referred to as system data storage 624). The O/S instruction(s) 622 and system data storage 624 can be immediately accessible to and/or are presently operated on by at least one processor of the processor(s) 602. The O/S instruction(s) 622 can embody an operating system for the user device 610. Specific implementation of such O/S can depend in part on architectural complexity of the user device 610. Higher complexity affords higher-level O/Ss. Example operating systems can include iOS, Android, Linux, Unix, Windows operating system, and substantially any operating system for a mobile computing device.

Memory 616 can comprise other removable/non-removable, volatile/non-volatile computer-readable non-transitory storage media. As an example, memory 616 can include a mass storage unit (not shown) which can provide non-volatile storage of computer code, computer readable instructions, data structures, program modules, and other data for the user device 610. A specific implementation of such mass storage unit (not shown) can depend on desired form factor of and space available for integration into the user device 610. For suitable form factors and sizes of the user device 610, the mass storage unit (not shown) can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), or the like.

The user device 610 can implement an IBM therapy and other functionalities of the mobile application 114 in accordance with aspects described herein by executing the mobile application 114. More specifically, in some embodiments, the IBM therapy and other functionalities can be implemented in response to execution of software components that constitute the mobile application 114 by at least one of the one or multiple processors 602.

In general, a processor of the one or multiple processors 602 can refer to any computing processing unit or processing device comprising a single-core processor, a single-core processor with software multithread execution capability, multi-core processors, multi-core processors with software multithread execution capability, multi-core processors with hardware multithread technology, parallel platforms, and parallel platforms with distributed shared memory (e.g., a cache). In addition or in the alternative, a processor of the group of one or more processors 408 can refer to an integrated circuit with dedicated functionality, such as an ASIC, a DSP, a FPGA, a CPLD, a discrete gate or transistor logic, discrete hardware components, or any combination thereof designed to perform the functions described herein. In one aspect, processors referred to herein can exploit nano-scale architectures such as, molecular and quantum-dot based transistors, switches and gates, in order to optimize space usage (e.g., improve form factor) or enhance performance of the computing devices that can implement the various aspects of the disclosure. In another aspect, the one or multiple processors 602 can be implemented as a combination of computing processing units.

The display device 604 can display the various user interfaces described herein in connection with an IBM therapy and other functionalities of the mobile application 114. In some embodiments, the display device 604 can be embodied in a touch display device. Accordingly, the display device 604 can include sensing arrays, such as arrays for capacity sensing, force sensing, or resistive sensing. The display device 604 also can include circuitry for determining touch points (e.g., a pressure points or a contact points) using electric signals from the sensing arrays. The display device 604 also includes display elements, such as pixels, light emitting diodes (LEDs), substrates, and the like. The display elements can be arranged in one or multiple layers having a spatial arrangement defined by the type of display device 604; namely, frontlit display or backlit display. The display device 604 also include a solid touch layer that interfaces with an end-user (e.g., participant 104).

The one or multiple I/O interfaces 606 can functionally couple (e.g., communicatively couple) the user device 610 to another functional element (a component, a unit, server, gateway node, repository, a device, or similar). Functionality of the user device 610 that is associated with data I/O or signaling I/O can be accomplished in response to execution, by a processor of the processor(s) 602, of at least one I/O interface retained in memory element 628. Such memory element being represented by the block labeled I/O interface(s) 628. In some embodiments, the at least one I/O interface embodies an application programming interface (API) that permit exchange of data or signaling, or both, via an I/O interface of I/O interface(s) 606. In some embodiments, the one or more I/O interfaces 606 can include at least one port that can permit connection of the user device 610 to another other device or functional element. In one or more scenarios, the at least one port can include one or more of a parallel port (e.g., GPIB, IEEE-1284), a serial port (e.g., RS-232, universal serial bus (USB), FireWire or IEEE-1394), an Ethernet port, a V.35 port, a Small Computer System Interface (SCSI) port, or the like.

The at least one I/O interface of the one or more I/O interfaces 606 can enable delivery of output (e.g., output data or output signaling, or both) to such a device or functional element. Such output can represent an outcome or a specific action of one or more actions described herein, such as action(s) performed in the example methods described herein.

The radio module 608 can send and/or receive wireless signals from a wireless device remotely located relative to the user device 610. The wireless signals can be sent and can be received according to a defined radio technology protocol wireless communication. The radio module 608 can include one or more antennas and processing circuitry that permit communicating wirelessly in accordance with the defined radio technology protocol. Thus, the radio module 608 can be configured to send and receive wireless signals according to one or several radio technology protocols including ZigBee™; Bluetooth™; near field communication (NFC) standards; ultrasonic communication protocols; or similar protocols. The antenna(s) and processing circuitry also can permit the radio module 608 to communicate wirelessly according to other radio technology protocols, including protocols for small-cell wireless communication and macro-cellular wireless communication. Such protocols include IEEE 802.11a; IEEE 802.1 lax; 3rd Generation Partnership Project (3GPP) Universal Mobile Telecommunication System (UMTS) or “3G;” fourth generation (4G); fifth generation (5G); 3GPP Long Term Evolution (LTE); LTE Advanced (LTE-A); wireless broadband (WiBro); and the like.

Although not shown in FIG. 6, the user device 610 can include a battery that can power components or functional elements within the user device 610. The battery can be rechargeable, and can be formed by stacking active elements (e.g., cathode, anode, separator material, and electrolyte) or a winding a multi-layered roll of such elements.

In addition to the battery, the user device 610 can include one or more transformers (not depicted) and/or other circuitry (not depicted) to achieve a power level suitable for the operation of the user device 610 and components, functional elements, and related circuitry therein. In some cases, the user device 610 can be attached to a conventional power grid to recharge the battery and ensure that the user device 610 and the functional elements therein can be operational. In one aspect, at least one of I/O interface(s) 606 can permit connecting to the conventional power grid. In some embodiments, the user device 610 can include an energy conversion component, such as a solar panel, to provide additional or alternative power resources or power autonomy to the user device 610.

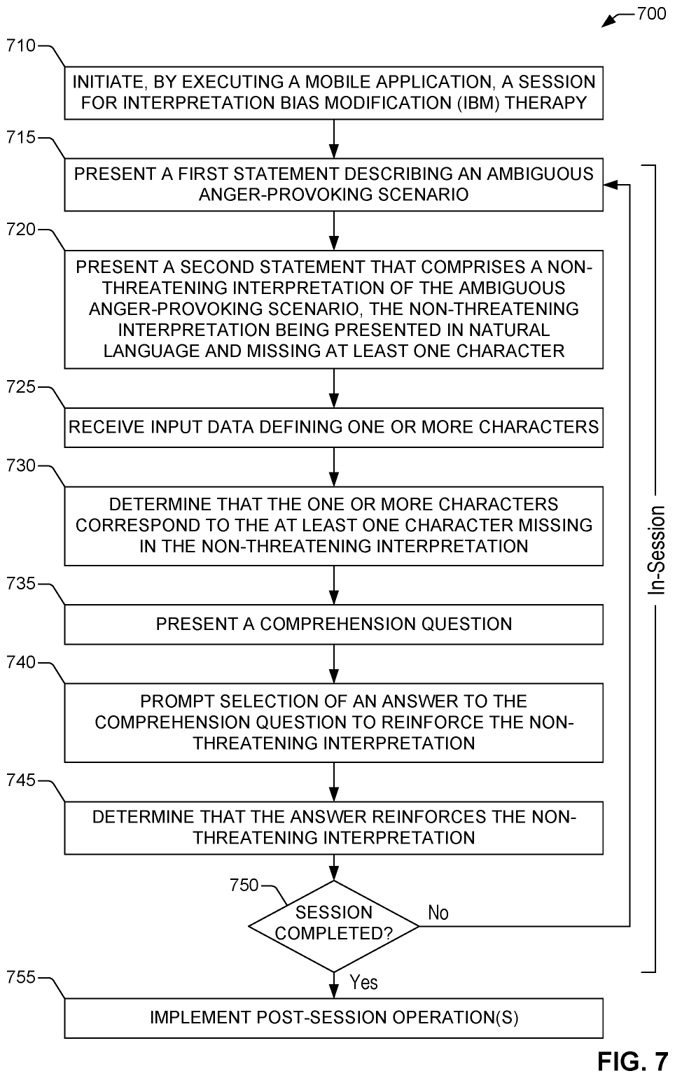

In view of the various aspects of the techniques disclosed herein, an example method that can be implemented in accordance with embodiments of this disclosure can be more readily appreciated with reference to the flowchart in FIG. 7. For purposes of simplicity of explanation, the example methods disclosed herein are presented and described as a series of blocks (with each block representing an action or an operation in a method, for example). However, it is to be understood and appreciated that the disclosed methods are not limited by the order of blocks and associated actions or operations, as some blocks may occur in different orders and/or concurrently with other blocks from that are shown and described herein. For example, the various methods or processes of the disclosure can be alternatively represented as a series of interrelated states or events, such as in a state diagram. Furthermore, not all illustrated blocks, and associated action(s), may be required to implement a method in accordance with one or more aspects of the disclosure. Further yet, two or more of the disclosed methods or processes can be implemented in combination with each other, to accomplish one or more functionalities and/or advantages described herein.

The methods of the disclosure can be retained on an article of manufacture, or computer-readable non-transitory storage medium, to permit or facilitate transporting and transferring such methods to a computing device for execution, and thus implementation, by a processor of the computing device or for storage in a memory thereof or functionally coupled thereto. Such a computing device can be embodied in a mobile computer, such as an electronic book reader (e-reader) or other tablet computers, or a smartphone; a mobile gaming console; or the like. In one aspect, one or more processors, such as processor(s) that implement one or more of the disclosed methods, can be employed to execute program instructions retained in a memory, or any computer- or machine-readable medium, to implement the one or more methods. The program instructions can provide a computer-executable or machine-executable framework to implement the methods described herein.

FIG. 7 illustrates an example of a method 700 for implementing an IBM therapy using a user device, in accordance with one or more embodiments of the disclosure. The user device can be embodied in a smartphone or a tablet computer, in some cases. The user device can implement, entirely or partially, the example method 700. To that end, the user device includes computing resources that can implement at least one of the blocks included in the example method 700. The computing resources include one or more processors or other types of processing circuitry; one or more memory devices or other types of storage circuitry; I/O interfaces; a combination thereof; or similar resources. In some embodiments, the user device can be embodied in the user device 610 (FIG. 6). In one example, the user device is embodied in or includes the mobile device 110.

At block 710, the user device can initiate a session for IBM therapy. The session is initiated by initiating execution of (or, in some cases, continuing executing) a mobile application (e.g., mobile application 114 (FIG. 1)).

At block 715, the user device can present, as part of the session, a first statement describing an ambiguous anger-provoking situation. In one example, the first statement can be the statement 124 (FIG. 1). The first statement can be presented within a user interface (e.g., user interface 120 (FIG. 1). That user interface can be presented by means of a display device that can be integrated into the user device, for example. Such a display device can be, for example, the display device 604 (FIG. 6).

At block 720, the user device can present, as part of the session, a second statement that comprises a non-threatening interpretation of the ambiguous anger-provoking situation. The second statement can be presented after the first statement. The non-threatening interpretation being presented in natural language and missing at least one character. The second statement can include the first statement and the non-threatening interpretation. As such, in one example, the second statement can include the statement 124 (FIG. 1) and the non-threatening interpretation 134 (FIG. 1). The second statement can be presented within a user interface (e.g., user interface 130 (FIG. 1). That user interface can be presented by means of the display device that can be integrated into the user device, for example.

At block 725, the user device, as part of the session, can receive input data defining one or more characters. The input data can be generated by a component of the user device in response to a user-interaction with the user device. As mentioned, the user-interaction can be one of a screen tap, a screen swipe, a screen click, or similar.

At block 730, the user device can determine, as part of the session, that the one or more characters correspond to the at least one character missing in the non-threatening interpretation. The user device can present a congratulatory message in response to determining that the at least one character correctly complete the character(s) missing in the non-threatening interpretation. The message can be presented in an overlay section on the user interface that presents the non-threatening interpretation (or, in some cases, a subsequent comprehension question). Continuing with the foregoing example, the non-threatening interpretation presented at block 720 can lack one character and that character can be the letter “d.” A single character can be received at block 725, and the user device can determine, in response to executing or continuing to execute the application, that the single character that has been received is the letter “d.” Further, in response to determining that the letter “d” has been received, the user device can be present a message including visual elements or aural elements, or both, that convey a congratulation (e.g., “Good job!” or “Well done!”). Such visual elements can be presented as an overlay section on the user interface that presents the non-threatening interpretation. In some cases, instead of presenting the overlay section, the visual elements can be included in such a user interface.

At block 735, the user device can present, as part of the session, a comprehension question in response to such a determination. The comprehension question corresponds to the non-threatening interpretation. Further continuing with the above example, the user device can present the comprehension question 144 (FIG. 1). The comprehension question can be presented within a user interface (e.g., user interface 140 (FIG. 1). That user interface can be presented by means of the display device that can be integrated into the user device, for example.

At block 740, the user device can prompt, as part of the session, selection of an answer to the comprehension question to reinforce the non-threatening interpretation. To that end, in some cases, the user device can present one or more selectable visual elements as part of the user interface that includes the comprehension questions. Such element(s) can constitute the prompt. The answer can be selected from a group of preset possible answers. For example, the user interface that includes the comprehension question can include a first selectable visual element and a second selectable visual element corresponding to respective preset possible answers to the comprehension question.

At block 745, the user device can determine, as part of the session, that the answer reinforces the non-threatening interpretation. For example, the user device can determine that the answer is a correct answer (e.g., a “Yes” answer) to the comprehension question. The user device can present a congratulatory message in response to the answer being correct. The message can be presented in an overlay section on the user interface that presents the comprehension question. In some cases, instead of presenting the overlay section, the message can be included as a part of the user interface the presents the comprehension question. In one example, the overlay section can include text, graphics, speech, and/or sounds conveying a congratulation, such as “Good job!” or “Well done!” In some embodiments, instead of performing such a determination, the user device can determine that an answer has been selected in response to the prompt at block 740, regardless of whether or not the selected answer is correct.

At block 750, the user device can determine if the session has been completed. In response to a negative determination (“No” branch), the user device can continue the session by presenting another scenario. To that end, flow of the example method 700 returns to block 715 where the user device can present another statement describing another ambiguous anger-provoking scenario. In the alternative, in response to an affirmative determination (“Yes” branch), flow of the example method 700 can continue to block 755 where the user device can implement one or several post-session operations. In some cases, the user device can implement a single post-session operation including terminating execution of mobile application. In other cases, the user device can implement the post-session operation(s) as part of continuing executing the mobile application. As part of implementing the post-session operation(s), in some embodiments, the user device can provide rewards for having completed the session. More specifically, the user device can generate a token representing completion of the session. The user device can then assign the token to a user profile corresponding to the mobile application (e.g., mobile application 114 (FIG. 1)).

Although not shown in FIG. 7, in some embodiments, the example method 700 can include other operations. In one embodiments, the user device can present a selectable visual element prompting the end-user to configure a session reminder; and also can present a user interface in response to selection of that selectable visual element. The user interface that is presented includes, for example, second selectable visual elements to configure the session reminder. In one example, the selectable visual element is the selectable visual element 420(4) (FIG. 4A) included in the user interface 400 (FIG. 4A), and the user interface that includes the second visual elements is the user interface 500 (FIG. 5), for example.

In addition, or in another embodiments, the user device can present a second selectable visual element prompting the end-user to complete a task; and also can present a second user interface in response to selection of the second selectable visual element, the second user interface comprising a selectable pane including one or more defined visual elements defining at least a portion of the task (e.g., a survey), where the selectable pane includes one or more second defined selectable visual elements to receive data responsive to an item of the task (e.g., the survey). In some cases, instead of presenting the second user interface, the user device can present a series of user interfaces constituting the task, where each of the user interfaces in that series can include selectable visual element(s) that permit receiving input data responsive to the task.

Further, or in yet another embodiment, the user device can cause presentation of a message periodically, the message prompting an end-user to maintain a current frequency of sessions for IBM therapy.

Additionally, or in still another embodiment, the user device can determine that a second session for IBM therapy has not been initiated for a defined time interval; and can then cause presentation of a message (e.g., a push notification) prompting the end-user to initiate the second session for IBM therapy.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

Throughout the description and claims of this specification, the words “include” and “comprise” and variations of the word, such as “including,” “comprising,” “includes” and “comprises,” mean “including but not limited to,” and are not intended to exclude other components, integers or steps. “Such as” is not used in a restrictive sense, but for explanatory purposes.

It is understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memristors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method, comprising:
initiating, by a user device having at least one processor, a session for interpretation bias modification (IBM) therapy, the session initiated by executing a mobile application;
presenting, by the user device, within the session, a first statement describing an ambiguous anger-provoking situation;
presenting, by the user device, within the session and after the first statement, a second statement that comprises a non-threatening interpretation of the ambiguous anger-provoking situation, the non-threatening interpretation being presented in natural language and missing at least one character;
receiving, by the user device, within the session, input data defining one or more characters;
determining, by the user device, that the one or more characters correspond to the at least one character missing in the non-threatening interpretation;
presenting, by the user device, within the session, a comprehension question corresponding to the non-threatening interpretation; and
prompting, by the user device, within the session, selection of an answer to the comprehension question to reinforce the non-threatening interpretation.

2. The method of claim 1, further comprising,
determining, by the user device, that the answer reinforces the non-threatening interpretation; and
continuing with the session, by the user device, by presenting a third statement describing a second ambiguous anger-provoking situation.

3. The method of claim 1, further comprising,
presenting, by the user device, a selectable visual element prompting the end-user to configure a session reminder; and
presenting, by the user device, a user interface in response to selection of the selectable visual element, the user interface including second selectable visual elements to configure the session reminder.

4. The method of claim 3, further comprising,
presenting, by the user device, a second selectable visual element prompting the end-user to complete a task; and
presenting, by the user device, a second user interface in response to selection of the second selectable visual element, the second user interface comprising a selectable pane including at least a portion of the task, wherein the selectable pane includes a defined element to receive data responsive to an item of the at least the portion of the task.

5. The method of claim 1, further comprising causing, by the user device, presentation of a message periodically, the message prompting an end-user to maintain a current frequency of sessions for IBM therapy.

6. The method of claim 3, further comprising,
determining, by the user device, that a second session for IBM therapy has not been initiated for a defined time interval; and
causing, by the user device, presentation of a message prompting the end-user to initiate the second session for IBM therapy.

7. The method of claim 1, further comprising,
determining, by the user device, that the session has been completed;
generating, by the user device, a token representing completion of the session;
assigning, by the user device, the token to a user profile corresponding to the mobile application.

8. A computing device, comprising:
at least one processor; and
at least one computer-readable non-transitory storage medium having computer-executable instructions stored thereon that, in response to execution by the at least one processor, cause the computing device to,
initiate a session for interpretation bias modification (IBM) therapy;
present, within the session, a first statement describing an ambiguous anger-provoking situation;
present, within the session and after the first statement, a second statement that comprises a non-threatening interpretation of the ambiguous anger-provoking situation, the non-threatening interpretation being presented in natural language and missing at least one character
receive, within the session, input data defining one or more characters;
determine that the one or more characters correspond to the at least one character missing in the non-threatening interpretation;
present, within the session, a comprehension question corresponding to the non-threatening interpretation; and
prompt, within the session, selection of an answer to the comprehension question to reinforce the non-threatening interpretation.

9. The computing device of claim 8, the at least one computer-readable non-transitory storage medium having further computer-executable instructions stored thereon that, in response to execution by the at least one processor, cause the computing device to, determine that the answer reinforces the non-threatening interpretation; and
continue with the session by presenting a third statement describing a second ambiguous anger-provoking situation.

10. The computing device of claim 8 comprising one or more memory devices containing multiple ambiguous anger-provoking situations including the first ambiguous anger-provoking situation and the second ambiguous anger-provoking situation.

11. The computing device of claim 8, the at least one computer-readable non-transitory storage medium having further computer-executable instructions stored thereon that, in response to execution by the at least one processor, cause the computing device to,
present a selectable visual element prompting the end-user to configure a session reminder; and
present a user interface in response to selection of the selectable visual element, the user interface including second selectable visual elements to configure the session reminder.

12. The computing device of claim 11, the at least one computer-readable non-transitory storage medium having further computer-executable instructions stored thereon that, in response to execution by the at least one processor, cause the computing device to,
present a second selectable visual element prompting the end-user to complete a task; and
present a second user interface in response to selection of the second selectable visual element, the second user interface comprising a selectable pane including at least a portion of the task, wherein the selectable pane includes an area to receive data responsive to an item of the at least the portion of the task.

13. The computing device of claim 8, the at least one computer-readable non-transitory storage medium having further computer-executable instructions stored thereon that, in response to execution by the at least one processor, cause the computing device to present a message periodically, the message prompting an end-user to maintain a current frequency of sessions for IBM therapy.

14. The computing device of claim 8, the at least one computer-readable non-transitory storage medium having further computer-executable instructions stored thereon that, in response to execution by the at least one processor, cause the computing device to,
determine that a second session for IBM therapy has not been initiated for a defined time interval; and
cause presentation of a message prompting the end-user to initiate the second session for IBM therapy.

15. One or more computer-readable non-transitory media storing processor-executable instructions that, in response to execution, cause a mobile device to perform operations comprising:
initiating a session for interpretation bias modification (IBM) therapy;
presenting, within the session, a first statement describing an ambiguous anger-provoking situation;
presenting, within the session and after the first statement, a second statement that comprises a non-threatening interpretation of the ambiguous anger-provoking situation, the non-threatening interpretation being presented in natural language and missing at least one character
receiving, within the session, input data defining one or more characters;
determining that the one or more characters correspond to the at least one character missing in the non-threatening interpretation;
presenting, within the session, a comprehension question corresponding to the non-threatening interpretation; and
prompting, within the session, selection of an answer to the comprehension question to reinforce the non-threatening interpretation.

16. The one or more computer-readable non-transitory media of claim 15, the operations further comprising,
determining that the answer reinforces the non-threatening interpretation; and
continuing with the session by presenting a third statement describing a second ambiguous anger-provoking situation.

17. The one or more computer-readable non-transitory media of claim 15, the operations further comprising,
presenting a selectable visual element prompting the end-user to configure a session reminder; and
presenting a user interface in response to selection of the selectable visual element, the user interface including second selectable visual elements to configure the session reminder.

18. The one or more computer-readable non-transitory media of claim 17, the operations further comprising,
presenting a second selectable visual element prompting the end-user to complete a task; and
presenting a second user interface in response to selection of the second selectable visual element, the second user interface comprising a selectable pane including at least a portion of the task, wherein the selectable pane includes an area to receive data responsive to an item of the at least the portion of the task.

19. The one or more computer-readable non-transitory media of claim 15, the operations further comprising causing presentation of a message periodically, the message prompting an end-user to maintain a current frequency of sessions for IBM therapy.

20. The one or more computer-readable non-transitory media of claim 15, the operations further comprising,
determining that a second session for IBM therapy has not been initiated for a defined time interval; and
causing presentation of a message prompting the end-user to initiate the second session for IBM therapy.
